(19) United States
(12) Patent Application Publication
(10) Pub. No.: US 2004/0138683 A1
(43) Pub. Date: Jul. 15, 2004

(54) Suture arrow device and method of using

(76) Inventors: Walter Shelton, Brandon, MS (US); Juha-Pekka Nuutinen, North Wales, PA (US)

Correspondence Address: KENYON & KENYON, 1500 K STREET, N.W., SUITE 700, WASHINGTON, DC, 20005, US

(21) Appl. No.: 10/338,685

(22) Filed: Jan. 9, 2003

(51) Int. Cl. A61B 17/04

(52) U.S. Cl. 606/151; 606/232

(57) ABSTRACT

The present invention provides suture arrow device for repairing tissue or attaching matter to tissue. More specifically, the present invention provides a suture arrow device comprising at least two implantation members connected by a connecting member.

### FIELD OF THE INVENTION

The present invention provides surgical implants for repairing tissue or attaching matter to tissue. More specifically, the present invention provides a suture arrow device comprising at least two implantation members connected by a flexible connecting member.

### BACKGROUND OF THE INVENTION

It has been shown that the fixation of meniscus traumas, like ruptures and lesions, by suturing with absorbable sutures gives better results than the removal of traumatized meniscal tissue, see e.g. N. A Palmeri, T. F. Winters, A. E. Joiner and T. Evans, “The Development and Testing of the Arthroscopic Meniscal Staple”, Arthroscopy, Vol. 5, No. 2, 1989, p. 156, hereby incorporated by reference. However, arthroscopic suturing is a complicated and tedious technique where risks for the patient are significant because of the danger of damaging vessels and nerves. Therefore, for a long time surgeons have desired an absorbable meniscus lesion fixation device, which has the advantages of absorbable suturing techniques but which may be used more rapidly and safely than sutures.

Several research groups have tried to develop absorbable meniscus lesion fixation devices, for example, Schreiber (U.S. Pat. No. 4,873,976), Winters (U.S. Pat. No. 5,059,206) and Tamminmáki et al. (U.S. Pat. No. 5,562,704) disclose arrow-like implants intended for the surgical repair of meniscal ruptures. However, the arrow-like implants have the disadvantage that the proximal end (stem or head) of the devices may cause tissue irritation and abrasion, particularly when placed in connection with the meniscus, because the stem or head may be left protruding from the outer surface of the meniscus.

Justin and Winters (U.S. Pat. No. 5,569,252) describe a fastener, an installation device, and a method for repairing tears in the soft tissue of a patient, including meniscal tears. The fastener has a variable-pitch helical protrusion along a central portion that decreases from the distal end to the proximal end and which may serve to bring the two sides of the tear into opposition as the fastener is advanced. However, this implant, which requires a screwing motion for installation, is slow and tedious to use arthroscopically and the turning of the implant through fibrous tissue, such as meniscus tissue, risks the fibrous tissue twisting around the implant thereby hindering or preventing the installation of the implant and/or damaging the tissue.

Grafton and Brunsvold (U.S. Pat. No. 6,056,778) describe a meniscal tissue repair device and an applicator to insert the device. The device has lateral grooves and the grooves disposed near the distal end of the device are angled to open proximally and the grooves disposed near the proximal end of the device are angled to open distally. The disadvantages of this device are the difficulty of inserting such a device accurately through a tear and also the grooves that are angled to open distally may cause damage to the tissue when the device is inserted.

Sikora et al. (U.S. Ser. No. 2002/0,019,649) describes a wound closure kit comprising: a needle, two anchors and a connecting member. The device, comprising two anchors and the connecting member, may be loaded inside the needle. The anchors, which are connected by a connecting member, may be inserted into the tissue and a pre-tied, sliding knot is tightened to tension the connecting member. However, the anchors are placed against the surface of the tissue, not entirely within the tissue, which may cause irritation.

Green (U.S. Pat. No. 6,190,401 B1) describes a device that consists of a pair of needles detachably secured to a pair of anchoring members having a plurality of barb-like projections extending outwardly therefrom and an apparatus to insert the device. The anchoring members are joined by a suture, which connects the ends of the anchoring members opposite the needles. The needles, which are fixed to the handle, can be moved only longitudinally. The disadvantage of this device is that the second anchoring member has to be inserted into a preset location.

Schreiber (U.S. Pat. No. 4,635,637) describes a surgical suture having a base member, two substantially parallel shafts upstanding from said base member and having pointed barbs at the ends thereof. In the described embodiments the base member is as thick as the shafts. Bowman (EP 1070 487 A2) describes a graft fixation device comprising, two implantation members connected by a connecting member. The implantation members have longitudinal passageways there through. Bowman and Bruker (U.S. Ser. No. 2001/0,029,382 A1) describe a fixation device comprising, two implantation members connected by a connecting member. The connecting member has at least one lateral wing member extending there form and the implantation members have longitudinal passageways there through. Sander (U.S. Pat. No. 5,269,783) and Sander (U.S. Pat. No. 5,374,268) describe a device that consists of a pair of needles detachably secured to a pair of anchoring members having a plurality of barb-like projections extending outwardly there from. The anchoring members are joined by a suture, which connects the ends of the anchoring members opposite the needles.

One problem with the above devices is that the longitudinal passages through the implantation members in EP 1070 487 A2 and U.S. Ser. No. 2001/0,029,382 A1 are for mounting prongs. Because of the passages, the implantation members must be relatively thick, which causes the need for large (traumatic) drill holes in the tissue into which the implantation members will be pushed. In U.S. Pat. No. 4,635,637, EP 1070 487 A2, U.S. Ser. No. 2001/0029382 A1, U.S. Pat. No. 5,269,783 and U.S. Pat. No. 5,374,268, the base and the anchoring or implantation members are inserted side by side at the same time. This feature makes the insertion device bulky and wide, which may make the insertion process difficult and increases the risk of operational trauma.

Oberlander (U.S. Pat. No. 5,702,462) describes a method of repairing a torn meniscus using anchoring members. Each anchoring member includes a dart and a suture attached to it. The darts may be inserted distal to the plane of the meniscus tear and then the sutures may extend across the plane of the tear and out of the meniscus, where the sutures are then tied together. Feagin and Glisson (U.S. Pat. No. 5,500,00) describe a device that consists of a suture anchoring member, a suture member and a suture retaining member. The barbed suture anchoring member and suture member may be inserted into the soft tissue repair site and across the tear and then the suture member may extend back through the original entry side of the tear. The retaining member may then be applied to the suture member followed by tensioning of the suture member. Schwartz et al. (U.S. Pat. No. 6,306,159 B1) describes a device comprising an outer wall anchor for engaging against an outside wall of the meniscus on a first side of a defect, and an inner meniscal anchor engaging an inner surface of the meniscus on a second side of the defect where the inner meniscal anchor has a locking mechanism and an adjustable suture connects the outer wall anchor to the inner anchor.

The disadvantage of the methods described in U.S. Pat. No. 5,702,462, U.S. Pat. No. 5,500,00 and U.S. Pat. No. 6,306,159 B1 is that they require a difficult arthroscopic knot tying, clipping or locking procedure and they also leave the tied knots, clips or locking mechanism on the surface or near the surface of the meniscus, which may damage the tissue.

The various demands upon these devices are high. For example, the device must be strong enough to maintain good contact with the lesion tissues after the operation so that rapid healing may occur. The device must also retain its strength long enough to allow for healing. The device should also not cause any damage to the cartilage surfaces of the femur and tibia. It must also be absorbed without causing complications that would prevent or hinder the healing of the lesion. Additionally, the installation of the device should be easy and rapid and should cause minimal operational trauma. Because of these high demands, the optimal absorbable meniscus lesion fixation device has not yet been developed.

Because of the limitations of other implants, a need exists for a bioabsorbable, partially bioabsorbable, or permanent fastener that allows a minimally invasive method for repairing a tear in soft or tough tissue and/or for the fixation of synthetic fibrous implants or living tissue transplants on or in living tissue.

A need also exists for such a fastener that is rapid and easy to install and gives a strong and safe fixation of the tissue tear, implant or transplant, and is minimally traumatic. The fastener may be made from a non-toxic, biocompatible polymer, polymer alloy or fiber reinforced polymer composite, specially designed to maintain its structural integrity during the healing of the tear and to prevent tissue abrasion.

A need also exists for a device that, once the implantation members are separately inserted, will leave only a thin and flexible part of the proximal, suture-like, part of the fastener on the surface of the tissue and does not protrude from the surface of the meniscus, when the meniscus is compressed under load (like during walking).

A need also exists for a device where the implantation members are loaded consecutively inside the insertion needle to reduce the needle size and thus minimize the operational trauma.

A need also exists for a device where the tension at which a rupture is closed can be easily adjusted during the insertion procedure. The tension adjustment is made possible when the implantation members are placed completely inside the tissue, the connecting member has a fixed length, and the technique and instrumentation allow the insertion of the second implantation member to be at an off-set location and at a depth that is not predetermined.

These and other objects may be attained with the fastener of the present invention.

### SUMMARY OF THE INVENTION

It is an object of the present invention to provide a fastener that allows a minimally invasive method for repairing a tear in soft or tough tissue and/or for fixation of synthetic fibrous implants or living tissue transplants on or in living tissue.

It is a further object to provide such a device that is rapid and easy to install and gives a strong and safe fixation of the tissue tear, implant or transplant, and is minimally traumatic and that may be made from a nontoxic, biocompatible polymer, polymer alloy or fiber reinforced polymer composite, specially designed to maintain its structural integrity during the healing of the tear and to prevent tissue abrasion.

It is a further object to provide a device in which the implantation members are completely inserted inside the tissue and only a part of the flexible connecting member, that connects the implantation members, is left on the surface of the tissue.

These and other objects are attained with the suture arrow device and method of use of the present invention.

In an embodiment of the present invention, a suture arrow device is provided, including a first tissue arrow, including a single body having at least one protrusion thereon and a pointed end, a second tissue arrow, including a single body having at least one protrusion thereon and a pointed end, and a flexible connecting member connecting the first and second tissue arrows. In another embodiment of the present invention, a method of repairing a body tissue rupture is provided. The method includes providing the suture arrow device of the present invention, inserting the first tissue arrow into the body tissue through said rupture, and inserting the second tissue arrow into the body tissue in an off-set location from the first tissue arrow, to close the rupture.

Another embodiment of the present invention includes a kit. The kit comprises a suture arrow device of the present invention, and an installation tool including a cannula and a pusher portion. In yet another embodiment of the present invention, a method is provided, including providing the kit described above where at least one of the suture arrow devices is loaded in the installation tool, inserting the installation tool into the body tissue at a first location, pushing the first implantation member using the pusher portion across the body tissue rupture, repositioning the installation tool to a second location, and pushing the second implantation member using the pusher portion into the body tissue.

Yet another method of the present invention includes, providing a suture arrow described above, positioning the first tissue arrow through the implant or transplant into tissue, passing the connecting member across the implant or transplant, and re-positioning the second tissue arrow into tissue at an offset location from the first implantation member.

### BRIEF DESCRIPTION OF THE DRAWINGS

FIG. 1 shows an embodiment of the suture arrow device in accordance with the present invention.

FIG. 2 shows another embodiment of the suture arrow device in accordance with the present invention.

FIG. 3 shows another embodiment of the suture arrow device in accordance with the present invention

FIG. 4 shows another embodiment of the suture arrow device in accordance with the present invention.

FIGS. 5A-5H show side views of embodiments of implantation members in accordance with the present invention.

FIGS. 6A-6F show examples of axial cross-sections of embodiments of implantation members of the present invention.

FIGS. 7A-7J show radial cross-sections of embodiments of implantation members of the present invention.

FIGS. 8A-8F show radial cross-sections of embodiments of connecting members of the present invention.

FIGS. 9A-9G show embodiments of ways to attach a connecting member to an implantation member of the present invention.

FIGS. 10 and 11 show known prior art views of the fibrous structure of meniscus tissue.

FIG. 12 shows the handle of an embodiment of a suture arrow insertion device of the present invention.

FIG. 13 shows two cannulated implantation members loaded on a metal spike and connected with a connecting member.

FIG. 14 shows an embodiment of the suture arrow device of the present invention loaded inside an embodiment of an insertion device usable with the present invention.

FIG. 15 shows a cross-section of meniscus tissue in which an embodiment of the suture arrow device of the present invention is being inserted.

FIG. 16 shows an embodiment of the present invention in which the first implantation member of the suture arrow device is inserted into the tissue.

FIG. 17 shows an embodiment of the present invention in which the insertion device is retracted after inserting the first implantation member.

FIG. 18 shows a top down view of an embodiment of the insertion device being repositioned.

FIG. 19 shows a top down view of meniscus tissue and the insertion device loaded with one implantation member after repositioning.

FIG. 20 shows a top down view of the suture arrow device inserted across a tear plane in the tissue where the connecting member is located partially on the surface of the tissue and the insertion device is retracted from the tissue.

FIG. 21 shows a top down view of an embodiment of the suture arrow device of the present invention inserted in the meniscus with a connecting member end cut.

FIGS. 22A-C show three methods of repairing a horizontal meniscus rupture using an embodiment of the suture arrow device in accordance with the present invention.

FIGS. 23A-B show the fixation of a fibrous mesh on the surface of living tissue by means of an embodiment of the suture arrow device of the present invention.

### DETAILED DESCRIPTION

The present invention provides a suture arrow device for repairing a tear in soft and/or tough tissue, such as a tear of the meniscus within the knee or a tear of the deltoid in the shoulder. The present invention also provides methods of using the suture arrow device provided.

The same identification numbers for the same elements are used throughout the Figures.

As is clear to one of ordinary skill in the art that the embodiments of the suture arrow device shown may have many different configurations and are not intended to be limited.

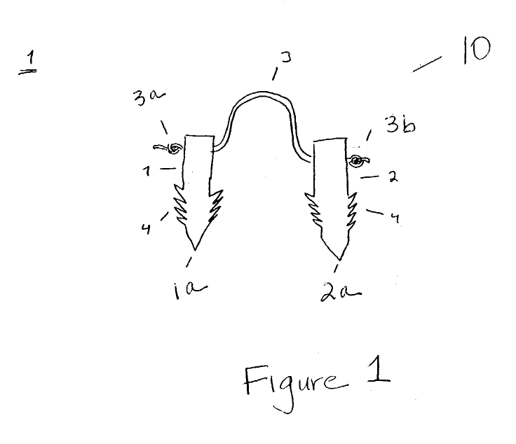

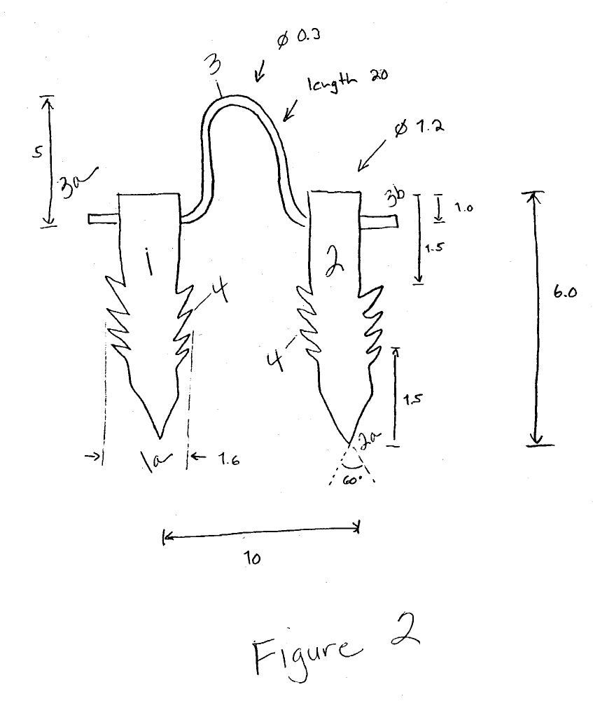

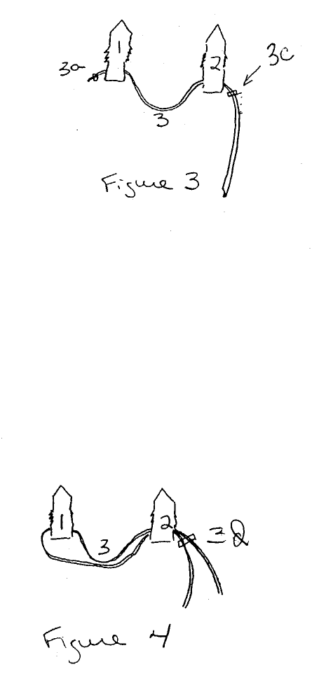

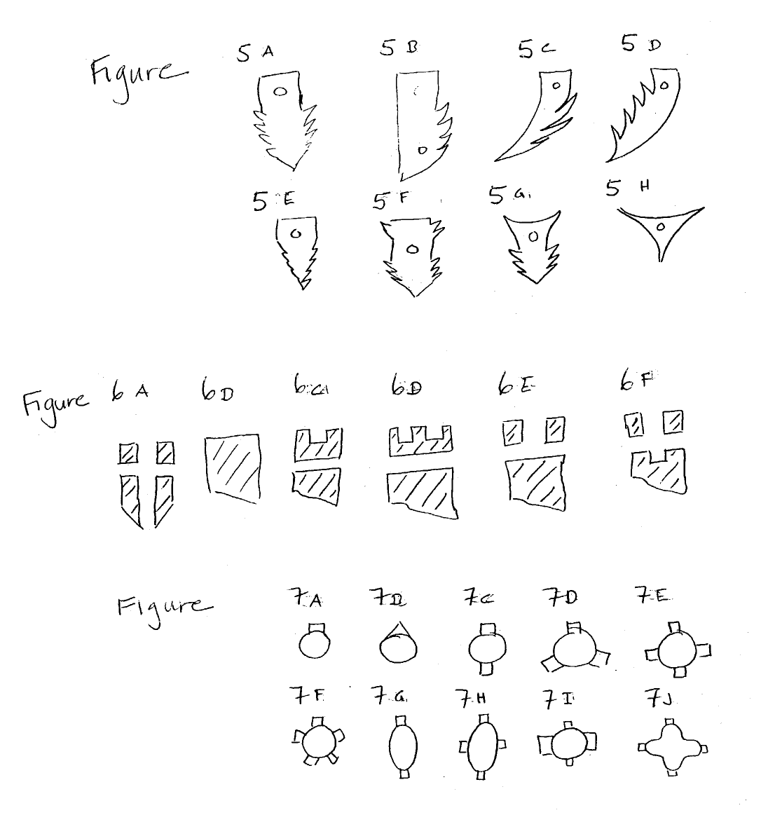

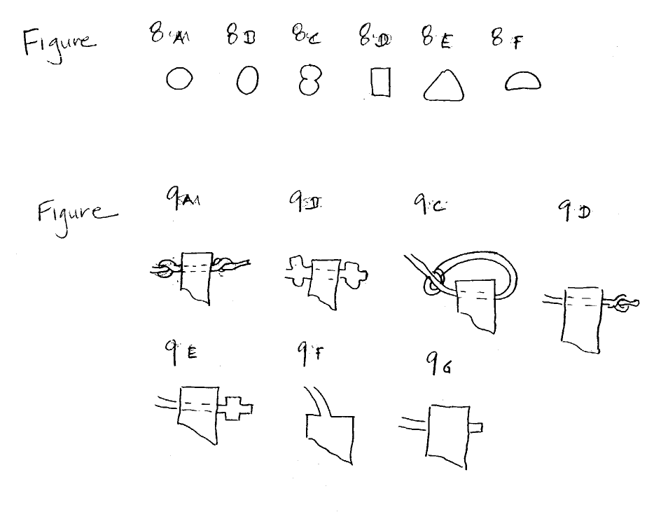

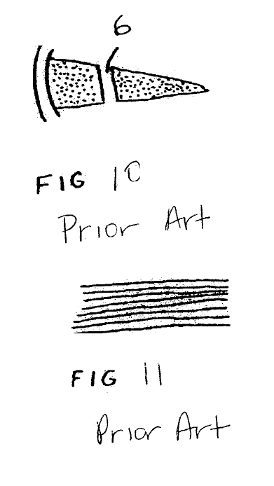

FIGS. 1 to 9 show embodiments of the suture arrow device of the present invention.

As shown in FIGS. 1 and 2, the suture arrow device 10 comprises two implantation members 1 and 2 and a connecting member 3. In the embodiments shown in FIGS. 1 and 2, the connecting member has a fixed length. In preferred embodiments the implantation members 1 and 2 are tissue fixation arrows and the connecting member is a braided or mono-filament suture. The suture material is either permanent or bioabsorbable. The connecting member could also be formed of a rubber or elastomer. Each of the implantation members has at least one barb 4 and each of their distal portions has a sharp tip 1a and 2a, respectively. The protrusions 4 shown are small, sharp-profile barbs, however, any known structure may be used, such as protruding ridges, pyramids, screw threads, or the like. In the embodiment of the present invention shown in FIGS. 1 and 2, the connecting member 3 may be threaded through holes in the implantation members 1 and 2 and the connecting member 3 may also have thicker portions 3a and 3b, such as knots. Although not shown, more than two implantation members may be attached to more than one connecting member, depending on the type of injury and the surgeon's desire. In a preferred embodiment, as shown in FIG. 2, the suture arrow device 10 is approximately 11 mm long, but may range from approximately 5 to 20 mm), including both the length of the implantation members 1 and 2 (approximately 6 mm each as shown in FIG. 2, but they may range from approximately 3 to 10 mm) and the length of the connecting member 3, which is approximately 5 mm as shown, but may range from approximately 2 to 10 mm. The diameter of each implantation member 1 and 2 is approximately 1.5 mm, but may range from approximately 1.0 to 3.0 mm) and the length of the connecting member 3 is approximately 20 mm, but may range from approximately 10 to 30 mm). The diameter of the connecting member is approximately 0.3 mm, but may range from 0.1 to 2.0 mm. The distance between implantation member 1 and 2, as measured from points 1a and 2a is approximately 10 mm, but may range from approximately 4 to 20 mm) and the points 1a and 2a form an approximate 60°, but may also be approximately a 30° to 90°) angle. In addition, the distance between two barbs 4 on a single implantation member (thus giving the approximate width of the implantation member) is approximately 2.2 mm and may range from approximately 1.2 to 3.6 mm). Finally, the location of the barbs 4 on a given implantation member, is approximately center and therefore approximately 0.5 to 4.0 mm from both the top and bottom of the implantation member, and the barb area is approximately 2 to 9 mm in length.

FIGS. 3 and 4 show additional embodiments of the present invention. In FIG. 3, a suture arrow device 10 is shown, which uses a sliding knot 3c instead of a fixed length connecting member 3. By using a sliding knot 3c, the suture arrow device may be more precisely adjusted to fit and secure a rupture or lesion in a tissue. The sliding knot 3c allows a surgeon to more easily correct the length of the connecting member so that the suture arrow device is secure.

FIG. 4 shows another embodiment of the suture arrow device where there is no pre-tied knot attaching the connecting member 3 to implantation member 1, instead, connecting member 3 is looped through implantation member 1 and then both ends of connecting member 3 may be looped through implantation member 2. The ends of connecting member 3 may then be formed into a sliding knot 3c or locking means 3d may be used to secure the ends. The locking means may include a deformable ring, which may be deformed and tightened around the connecting member ends or a locking collar. The locking means may also be used with a single end of a connecting member 3 as shown in FIG. 3.

FIGS. 5A to 7J, show different embodiments of implantation member configurations.

As shown in FIGS. 5A-5H, an implantation member may have a cylindrical body and a tapered tip, as in FIG. 5A, or the implantation member may have a conical body, as in FIG. 5E. The barbs may only be on one side of the implantation member, as in FIGS. 5B and 5C, and the implantation member may be straight or curved, as in FIGS. 5B-5D. The attachment of a connecting member may be at either end of the implantation member or in the middle, as in FIGS. 5A, 5B and 5F. The end portion of the implantation member may the shaped to resist movement opposite to the installation direction, as in FIG. 5G. Finally, the implantation member may have only one row of barbs and a tapered tip, as in FIG. 5H.

The implantation member may also have at least one axial recess to accommodate the tip of an insertion device. The recess may go totally or partially through the implantation member, as shown FIGS. 6A and 6C. If the implantation member has a hole through the shaft for the connecting member, the axial recess or recesses may come together with that hole, may not, or may go through the hole, as in FIGS. 6D-6F.

The protuberances/protrusions 4 may be located on the surface of the implantation members 1 and 2 of the device 10. The protrusions 4 are typically barbs, scales, threads, serrations, ridges or the like. These protrusions 4 prevent the installed suture arrow device 10 from slipping or moving out of the tissue in the (proximal) direction opposite to the direction of installation.

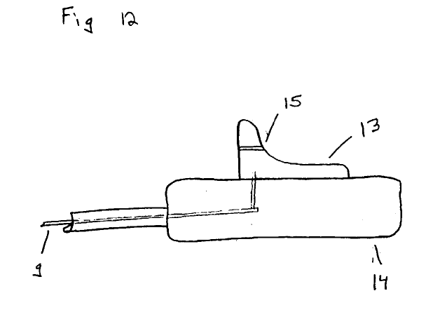

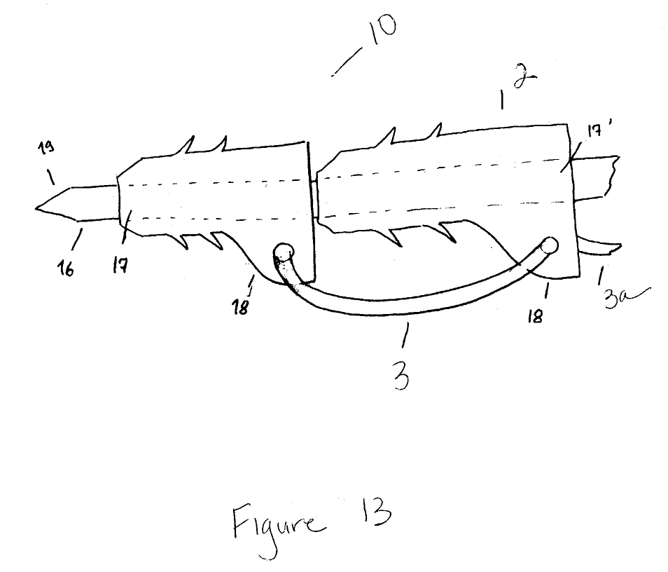

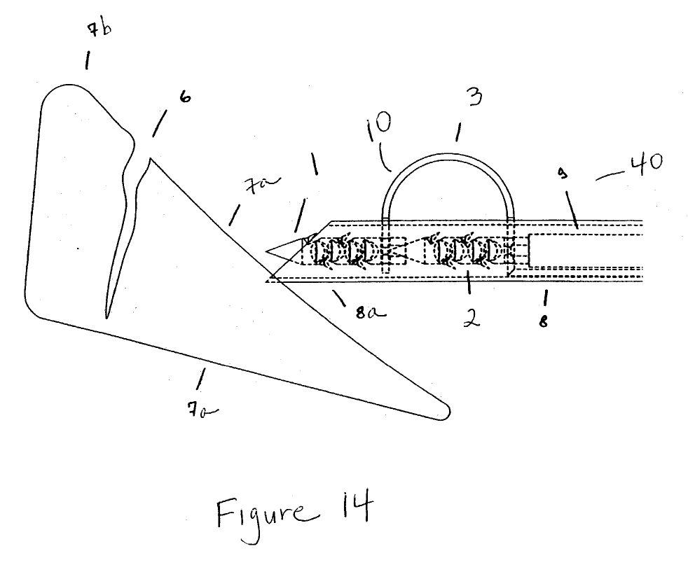

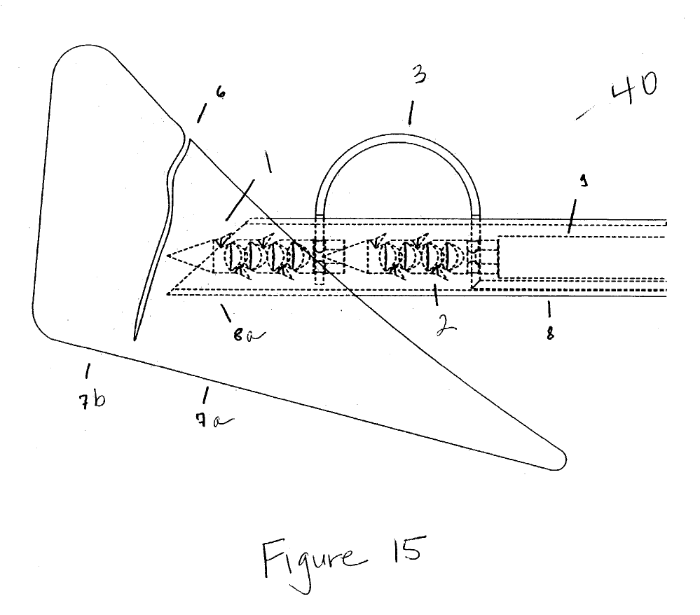

The barbs 4 of the implantation member may be rectangular or triangular shape, as in FIGS. 7A and 7B. The barbs may be only on one side of the implantation member, as in FIGS. 7A and 7B, on two sides, as in FIG. 7C, on three sides, as in FIG. 7D, on four sides, as in FIG. 7E, or on five sides, as in FIG. 7F. The cross-sectional geometry of the implantation member may also be oval, as in FIGS. 7G and 7H or axially grooved, as in FIG. 7J. The barbs may also have different sizes, as in FIG. 7I.

In order to lock the suture arrow 10 in the tissue, at least one or more of the implantation members 1 and 2 must penetrate the rupture plane inside the tissue and at least one or more of the protrusions 4 must also penetrate the rupture plane inside the tissue. In addition, the tapered sharp form of the tips 1a and 2a of implantation members 1 and 2 allows easy, minimally traumatic penetration of the suture arrow device 10 into the tissue. The protrusions 4 further allow for the locking of the implantation members 1 and 2 into the meniscal tissue when the suture arrow device is pushed, shot or hammered into the tissue. When completely installed, only a small, flexible, loop of the connecting member remains on the tissue surface. The connecting member that remains partially on the tissue surface may have a fixed length and it thus makes it possible to create an adjustable compression between the tear sides of the tissue when the latter implantation member is pushed or shot into the tissue with the delivery (installation) tool. The insertion of the latter implantation member is achieved by pushing the implantation member forward gradually until a desired tension is achieved or by pushing the implantation member forward using a preset force (shooting). This compression serves to close the rupture and may promote healing. In addition, because the suture arrow may be located mainly inside of the tissue the risks of prior devices may be eliminated. For example, the complications originating from (a) the presence of the bulky proximal part of the device on the meniscal surface, or (b) the cutting of collagen fibers inside of meniscus by the first (proximal) protrusions may no longer be issues.

Therefore, the combined effect of the barbed implantation members 1 and 2 and the connecting member 3 may be to lock the suture arrow device effectively in the tissue to close, fixate, and enhance the healing of the rupture. In a preferred embodiment the protrusions of the implantation members of the device are formed so that they do not prevent the device from being inserted into the tissue (in the distal direction) but do resist the device from slipping in the (proximal) direction, which is opposite to the installation direction.

In addition to the protrusions 4, the surface of the implantation members 1 and 2 may also include longitudinal ridges. The ridges may promote healing of the rupture by providing channels through which beneficial blood flow may occur along the length of the suture arrow device 10. These channels, which are typically about 0.05-0.5 mm wide and deep, act as capillaries, transporting blood from the highly vascularized distal portion of the tissue to the poorly vascularized proximal portion of the tissue.

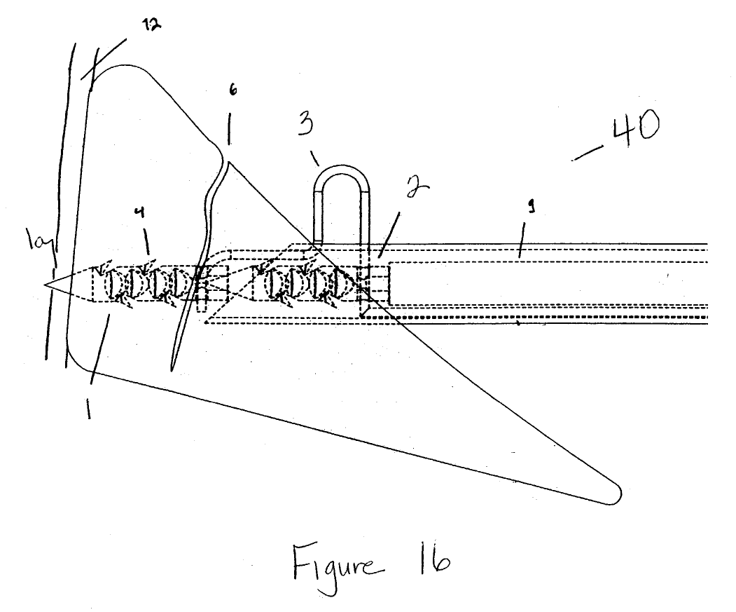

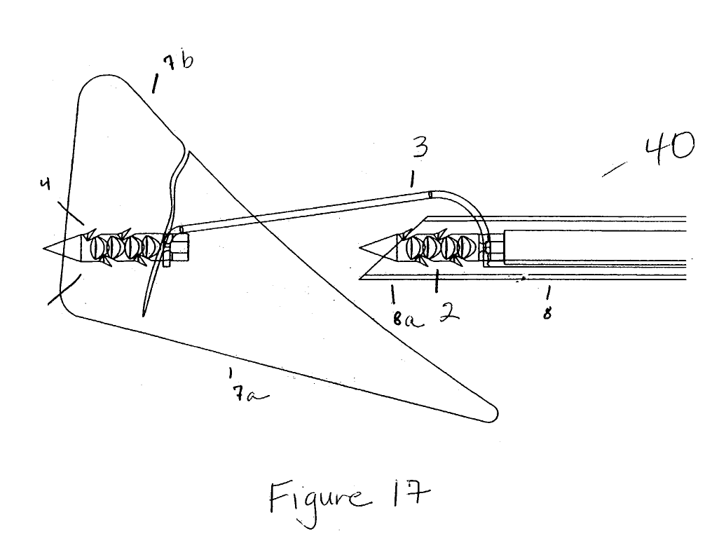

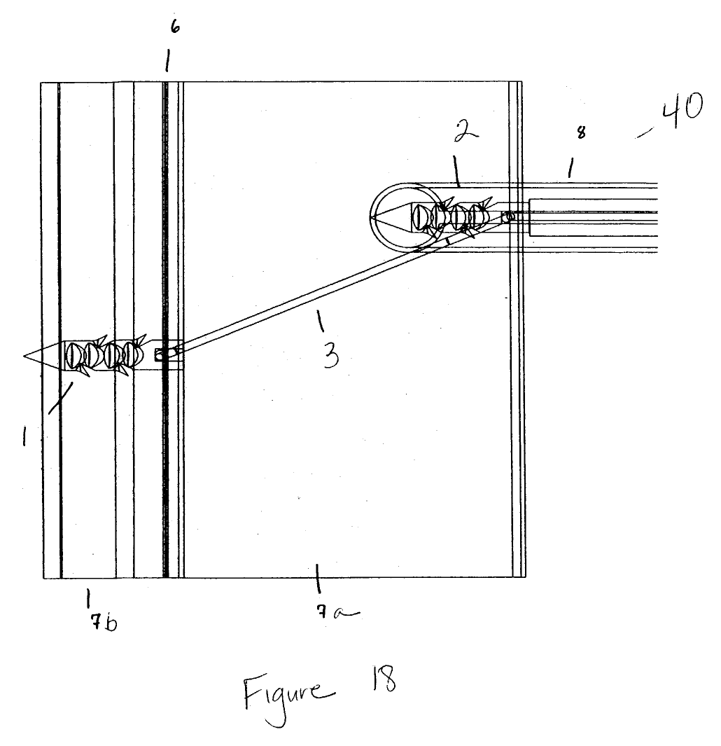

FIGS. 8A-8F show additional embodiments of structures for the connecting member 3. The connecting member 3 may be attached to the implantation members 1 and 2 in any number of ways, for example, as shown in FIGS. 9A-9G.

The connecting member may be attached to the implantation members by using adhesive or thermal forming, as shown in FIGS. 9F and 9G. The connecting member may also be threaded through a hole in the shaft in the implantation member, as shown in FIGS. 9A-9E. The connecting member may have a thick portion on both sides or on one side of the implantation member to hold an end of the connecting member, as shown in FIGS. 9B and 9E and the thick portions may be knots, as shown in FIGS. 9A, 9C and 9D.

In a preferred embodiment of the present invention, the suture arrow device may be installed in the meniscus tissue of the knee. FIGS. 10 and 11 show two different view of meniscus tissue. FIG. 10 shows a view of the meniscus with a tear 6 visible. FIG. 11 shows the internal collagen fiber structure of a meniscus from the direction of insertion along the long axis of a suture arrow device of the present invention. In FIG. 11, the collagen fibers are seen as parallel, horizontal fiber bundles.

It is typical that the microstructure of a meniscus contains reinforcing collagen fibers. Inside of a meniscus, many collagen fibers are oriented in a horizontal plane nearly parallel to the lower surface of the meniscus. If the horizontal collagen fibers are examined in a cut cross-section of a meniscus (as shown in FIG. 10) their cut ends may be seen microscopically as points on the cross-sectional surface. The typical vertical meniscus-lesion (rupture) 6 develops along the long axes of collagen fibers, because the binding forces between collagen fibers are weaker than along the long axis of fibers.

Because of the special arrangement of main portion of reinforcing horizontal collagen fibers inside of the meniscus, shown schematically in FIGS. 10 and 11, it is advantageous that the protrusions 4 of implantation members 1 and 2 are located at least on their upper and/or lower surfaces, so that as the implantation members, 1 and 2, penetrate into the meniscal tissue, the distal protrusions 4 slide forward through the collagen fiber bundles and grab finally between the horizontal collagen fiber bundles, locking the fastener in place.

It is well known that the meniscus also includes oriented fibers that are not horizontal. For example, the meniscus may also contain fibers having radial or oblique orientations. The collagen fibers form an essentially three-dimensional network in the meniscus, with such fibers being of particular importance with regard to using the present invention for treating the typical vertical (bucket handle) tears that occur.

To install a suture arrow device of the present invention, an installation tool may be used. FIG. 12 shows a handle portion 14 of an embodiment of such an installation tool. The handle portion 14 includes a trigger mechanism 13, which initiates deployment of a suture arrow device. The trigger mechanism 13 may be attached to an insertion pusher portion 9. The trigger 13 includes an opening 15 where the proximal tail of the suture 3 is attached.

FIG. 13 shows another embodiment of a device that may be used to insert a suture arrow device of the present invention. FIG. 13 shows a suture arrow device 10, including two implantation members 1 and 2 connected by connecting member 3 through holes 18. Each implantation member 1 and 2 has an axial hole 17 through it. A spike 16 is used to hold the two implantation members 1 and 2. The spike 16 is fed or threaded through holes 17 and the spike has a smaller diameter than the inner diameter of holes 17. The spike 16 has a pointed end 19, which allows for increased ease in installing the suture arrow device in tissue.

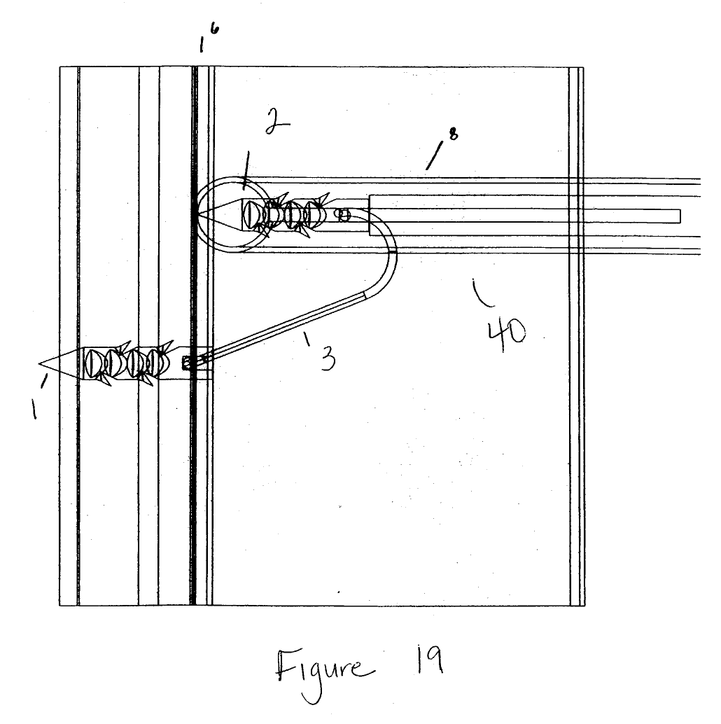

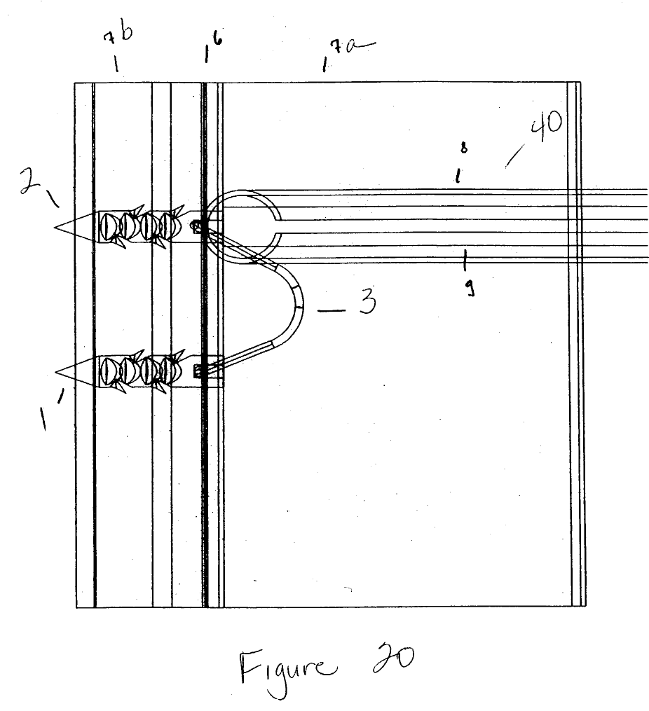

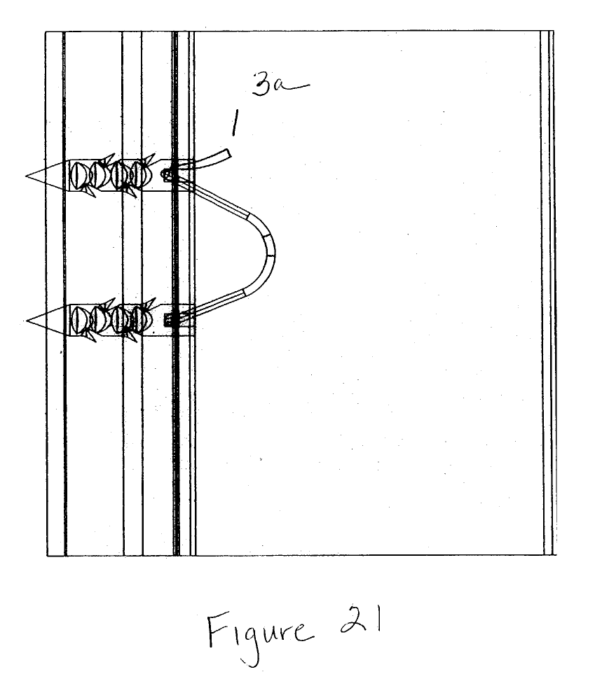

FIGS. 14 to 21 show an embodiment of a method of inserting a suture arrow device of the present invention. The method shown in FIGS. 14-21 may be used in any tissue, but a preferred tissue type is that of the meniscus of the knee and FIGS. 14-21 will be described with reference to the meniscus.

A preferred method of inserting the suture arrow device comprises two consecutive phases. First, one implantation member is inserted into the tissue across the tear or rupture in the tissue using an installation tool. Second, the installation tool is removed from the tissue and moved/re-positioned to another location, where a second implantation member is inserted into the tissue at a second location, so that the second implantation member is also situated across the tear. Following installation, the installation tool is removed. The length of the connecting member determines the distance and depth to which the implantation members may be inserted. In an embodiment of the present invention, part of the connecting member may remain on the surface of the tissue during the final stage of installation. In a preferred embodiment, where the tissue is the meniscus, using an appropriate level of force, the second implantation member may be forced deep into the meniscus so that part of the connecting member is located at the bottom of a small notch on the surface of meniscus. In this case, no disturbance to the opposite joint cartilage surface of the distal joint surface of the femur occurs.

As indicated above, FIGS. 14-21 show a preferred embodiment of the method of inserting a suture arrow device of the present invention. In FIG. 14, a meniscus with a rupture 6, separating the meniscus into a proximal side 7a and a distal side 7b, is shown. After loading at least one suture arrow device 10 into an installation tool 40, the tip 8a of an installation cannula 8 is pushed into a knee joint through a small incision (not shown). The tip 8a is located on the surface of the proximal part of the meniscus 7a (in relation to the rupture 6). It should be noted that more than one suture arrow device may be loaded in installation tool 40 and/or a suture arrow device including multiple installation members and connecting members may be loaded. In the case of more than two implantation members, the total length of all the connecting members connecting the implantation members is fixed, but the length of individual connecting member may be adjustable.

FIG. 15 shows the rupture 6 being reduced or compressed as the tip 8a of the installation cannula 8 is pushed into the meniscus. In the reduced rupture 6, the proximal 7a, and distal 7b rupture sides move closer to each other. The insertion pusher 9 within the cannula 8 keeps the first implantation member 1, the second implantation member 2, and the connecting member 3 from moving proximally when the tip 8a of the installation cannula 8 is pushed into proximal side 7a of the meniscus.

As seen in FIG. 16, insertion pusher 9 moves to the left (distally) and pushes the second implantation member 2, which pushes the first implantation member 1 at least partially through the reduced rupture 6. The distal movement of the insertion pusher 9 is limited by way of, e.g. a stopper (not shown). The tip 1a of the first implantation member 1, may protrude into the capsular tissue 12.

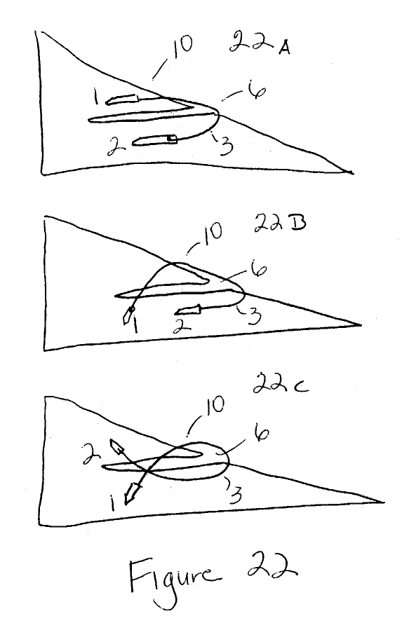

FIG. 17 shows the installation cannula 8 being retracted from the proximal side 7a of the meniscus. The first implantation member 1 stays in the distal side 7b of the meniscus, because of the proximally angled barbs 4 on the shaft. The installation cannula 8 moves to the right (proximally) and creates tension on the connecting member 3 to verify the locking of the first implantation member 1. The second implantation member 2 stays inside the installation cannula 8 during the tensioning of the connecting member 3, because of the (a) stoppers (not shown) located at the tip 8a of the installation cannula 8 and (b) suture member 3 that connects the second implantation member 2 to a trigger 13 in handle 14 as shown in FIG. 12.

FIG. 18 shows as viewed from the top looking down on the meniscus, the retracted installation cannula 8. The connecting member 3 is coming out of the proximal side 7a of the meniscus. The installation cannula 8 is repositioned on the surface of the meniscus 7a to insert the second implantation member 2. The placement of the second implantation member 2 is restricted by the length of the connecting member 3 and the insertion depth of the first implantation member 1. The flexible connecting member 3 and the small installation cannula 8 make it possible to place the second implantation member vertically or horizontally in respect to the first implantation member 1 depending on need.

FIG. 19 shows the installation cannula 8 repositioned horizontally from the first implantation member 1 and the installation cannula 8 pushed into the meniscus. In a preferred embodiment, both of the implantation members 1 and 2 are inserted through the reduced rupture 6, however, a fixation of the rupture may be achieved by inserting only one of the implantation members through the rupture 6.

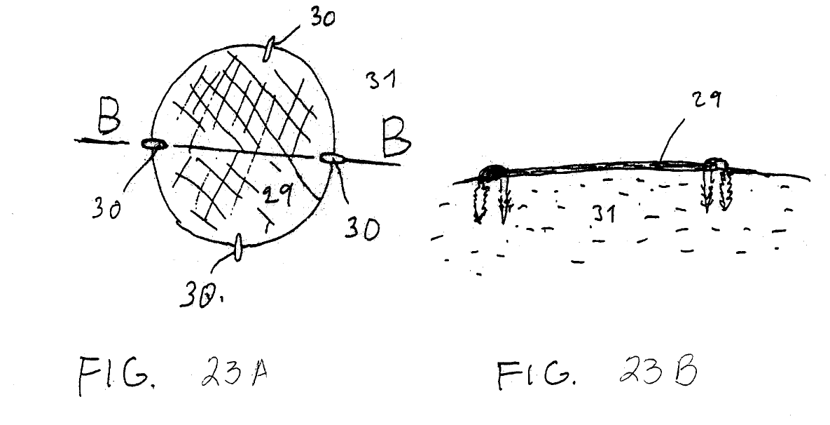

FIG. 20 shows the insertion of the second implantation member 2. The distally (left in FIG. 20) moving insertion pusher 9 inside the installation cannula 8 pushes the second implantation member 2 into the meniscus tissue. While the second implantation member 2 moves distally, it pulls the connecting member 3 along and this creates tension on the connection member 3, which creates the pulling force into the proximal part of meniscus 7a, tightening the rupture 6 and creating a compression force between the distal 7b and proximal 7a sides of the meniscus. The tension on the connecting member 3 leaves it partially on the small notch of the meniscal surface.

The connecting member 3 that comes out of the meniscus, is released from the opening 15 in the handle 14 and the installation cannula 8 and the insertion pusher 9 are retracted from the meniscus tissue leaving the second implantation member 2 at the position where it was pushed. The suture member 3 is threaded through a suture cutter, the suture cutter is inserted into the joint, slid distally on the proximal surface of the meniscus and the suture member 3 is cut as short as possible, as shown in FIG. 21. In an advantageous embodiment, the implantation members, 1 and 2, are located vertically i.e. the connecting member 3 creates a vertical loop over the horizontal collagen fibers.

FIGS. 22A, 22B and 22C illustrate a preferred method of installing the fastener to repair a rupture 6 in a meniscus tissue. In the rupture 6, a plane of rupture is horizontal and it may be reduced and repaired by placing one implantation member 1 above and one implantation member 2 under or below the rupture plane 6, as shown in FIG. 22A. The horizontal rupture 6 may also be reduced and repaired by placing one or more implantation members 1 and 2 through the rupture plane, as shown in FIGS. 22B and 22C.

In addition to using the suture arrow device of this invention in securing tears or closing wounds in living tissues, the suture arrow devices may be applied to fixate synthetic fibrous implants, like membranes, meshes, non-woven felts, fibrous scaffolds, etc. on or in living tissues. Such synthetic fibrous implants are described e.g. in EPO Pat. No. 0423155, U.S. Pat. No. 6,007,580 and PCT/EP 98/03030.

When using the suture arrow devices of this invention in fixation of synthetic fibrous implant or biological transplant on or into living tissue, the implant or transplant is first aligned on the surface or inside the living tissue. Thereafter, suture arrow devices are inserted one after another through or off-set from the implant or transplant so that the distal barbed parts of the implantation members lock the suture arrow into the living tissue below the implant or transplant and the connecting member remains on the surface of the implant or transplant securing it to the surface (or inside) of the living tissue. FIG. 23A shows, as seen from above, and FIG. 23B as seen from the side in plane B-B of FIG. 23A, how a fibrous mesh 29 has been secured with suture arrows 30 on a living tissue 31. Typical living tissue transplants, which may be fixed with the devices of this invention, are autografts, allografts and xenografts, like collagen membranes and felts, periosteum transplants or connective tissue transplants.

The devices, including the implantation members and the connecting members of this invention may be manufactured of bioabsorbable polymers, copolymers or polymer mixtures or alloys with melt molding methods known in the prior art. It is also possible to use the techniques of U.S. Pat. No. 4,743,257, hereby incorporated by reference, to mold in a compression or injection mold absorbable fibers and binding polymer together to create a fiber-reinforced or especially a self-reinforced structure. The devices of this invention may be molded in a single compression molding cycle, or the protrusions may be machined on the surface of a device after the molding cycle.

The oriented and/or self-reinforced structure may also be created during extrusion or injection molding of absorbable polymeric melt trough a suitable die or into a suitable mold at high speed and pressure. When cooling occurs at suitable conditions, the flow orientation of the melt remains in the solid material as an oriented or self-reinforcing structure. In an advantageous embodiment, the mold may have the form of the device, but it is also possible to manufacture the devices of the invention by machining (possibly using heat) and thermoforming (e.g. by bending the proximal end) of injection-molded or extruded semi-finished products

It is advantageous to make the devices of melt-molded, solid-state drawn or compressed, bioabsorbable polymeric materials, which are described e.g. in U.S. Pat. No. 4,968,317 or 4,898,186, both hereby incorporated by reference.

The reinforcing fibers of the device may also be ceramic fibers, like bioabsorbable hydroxyapatite or bioactive glass or tricalcium phosphate fibers. Such bioabsorbable, ceramic fiber reinforced materials are described e.g. in European Patent Application No. 0146398 and in WO 96/21628.

The oriented and/or self-reinforced or otherwise fiber reinforced devices of this invention may be manufactured by molding the reinforcement fiber-polymer matrix to the final product in a mold, whose mold cavity has the form of the final product or the final form may be machined mechanically (possibly also using heat) on a preform, such as a melt-molded and solid-state drawn rod, as is described e.g. in U.S. Pat. No. 4,968,317.

The reinforcement elements may extend into any protrusions or ridges of the device. The reinforcement elements may also turn spirally around the long axis of the implantation members and/or of the connecting member. Also other different orientations of reinforcement elements in elongated samples, which are familiar from composite technology, may be applied to the present invention. However, a general feature of orientation and/or fiber-reinforcement or self-reinforcement of the devices of this invention is that many of the reinforcing elements are oriented in such a way that they may carry effectively the different external loads (such as tensile, bending and shear loads) that are directed to the healing rupture (for example loads to a meniscus caused by the movements of the patient's knee).

According to an advantageous embodiment of the invention, the suture arrow device, or a special coating layer on its surface, may contain one or more bioactive substances, such as antibiotics, chemotherapeutic substances, angiogenic growth factors, substances accelerating the healing of the wound, growth hormones and the like. Such bioactive meniscal repair devices are especially advantageous in surgical use, because they chemically contribute to the healing of the lesion in addition to providing mechanical support.

The oriented and/or reinforced materials of the devices typically have initial tensile strengths of 100-2000 MPa, bending strengths of 100-600 MPa and shear strengths of 80-400 MPa. Additionally, they may be made stiff and tough or flexible. These mechanical properties are superior to those of non-reinforced absorbable polymers which typically show strengths between 40 and 100 MPa and may additionally be brittle (see e.g. Ref. 3 S. Vainionp\acute{aa}, P. Rokkanen and P. Tórmálá, “Surgical Applications of Biodegradable Polymers in Human Tissues”, Progr. Polym. Sci 14/1989, pp. 679-716).

The devices of the present invention may be sterilized by any of the well known sterilization techniques, depending on the type of material used in manufacture of the device. Suitable sterilization techniques include heat or steam sterilization, radiation sterilization such as cobalt 60 irradiation or electron beams, ethylene oxide sterilization, and the like.

After the description above of the present invention and certain specific embodiments thereof, it will be readily apparent to those skilled in the art that many variations and modifications may be made to the present invention without departing from the spirit and scope thereof.

The principles of the present invention described broadly above will now describe with reference to the following specific example, without intending to restrict the scope of the present invention.

### EXAMPLE 1

A cylindrical, continuous billet was extruded from PLA 96L/4D polymer (i.v.≈6.5, manufacturer: Purac Biochem B.V., Holland) with a single screw extruder (Extrudex, φ115 mm). The billet was drawn in the solid state (at temperature of 105-110° C.) to a draw ratio of 4. The drawn billet had a diameter of 1.2 mm and was cut into pieces of 6 mm in length. The tips of the formed implantation members were sharpened and barbs were cut on four sides of the implantation members. A hole having a diameter of 0.4 mm was drilled through the shaft and an axial recess having a diameter of 0.5 mm and depth of 0.5 mm was drilled to the end of the shaft that was not sharpened. A braided 2-0 suture was threaded through two shafts and a knot tied on both sides of each shaft.

The feasibility of the suture arrow was tested using porcine meniscus. The suture arrow was implanted into the menisci using arthroscopic prototype instrument, which consisted of a cannula part and a pusher part. The insertion of the suture arrow was simple and the fixation achieved by doing the insertion in two consecutive phases was good. The pullout force of the device was tested by pulling from the connecting member and the force was comparable to those reported in the literature for other meniscus repair devices.

We claim:

1. A suture arrow device comprising:
a first tissue arrow, including a single body having at least one protrusion thereon and a pointed end;
a second tissue arrow, including a single body having at least one protrusion thereon and a pointed end; and
a flexible connecting member connecting the first and second tissue arrows.

2. The suture arrow according to claim 1, wherein the connecting member is a mono-filament or braided suture.

3. The suture arrow according to claim 1, wherein the connecting member has a fixed length.

4. The suture arrow according to claim 1, further comprising means for locking the connecting member.

5. The suture arrow according to claim 1, wherein the protuberances of the first and second tissue arrows are selected from the group consisting of transverse ridges, barbs, pyramids, threads, and combinations thereof.

6. The suture arrow according to claim 1, wherein the first and second tissue arrows are longitudinally drawn and oriented.

7. The suture arrow according to claim 1, wherein the first and second tissue arrows are bioabsorbable.

8. The suture arrow according to claim 1, wherein the connecting member is bioabsorbable.

9. The suture arrow according to claim 1, wherein the connecting member has a flexible structure comprising rubber or an elastomer.

10. The suture arrow according to claim 1, further comprising at least one or more additional tissue arrows and connecting members.

11. The suture arrow according to claim 1, wherein the suture arrow comprises a bioactive material.

12. The suture arrow according to claim 1, wherein the first and second tissue arrows are cannulated.

13. The suture arrow according to claim 12, wherein the first and second tissue arrows are capable of being inserted into a tissue on a metal spike.

14. A method of repairing a body tissue rupture comprising:
providing the suture arrow of claim 1;
inserting a first tissue arrow into the body tissue through a rupture; and
inserting a second tissue arrow into the body tissue in an off-set location from the first tissue arrow, to close the rupture.

15. The method of claim 1, wherein inserting the second tissue arrow further comprises moving the second tissue arrow to an off-set location.

16. The method of claim 15, wherein moving further includes tensioning the connecting member.

17. A kit comprising:
at least one of the suture arrow device of claim 1; and
an installation tool including a handle, a cannula, and a pusher portion.

18. The kit of claim 17, wherein the installation tool further includes a trigger mechanism.

19. A method of repairing a body tissue rupture comprising:
providing the kit of claim 17, wherein at least one of said suture arrow devices is loaded in said installation tool;
inserting the installation tool into the body tissue at a first location;
pushing the first implantation member using the pusher portion across the body tissue rupture;
re-positioning the installation tool to a second location; and
pushing the second implantation member using the pusher portion into the body tissue.

20. The method of claim 19, wherein the second implantation member also is pushed across the body tissue rupture, at an off-set location.

21. A method for the fixation of a fibrous implant or a tissue transplant, on or in a living tissue comprising:
providing the suture arrow of claim 1;
positioning the first tissue arrow through the implant or transplant into tissue;
passing the connecting member across the implant or transplant; and
re-positioning the second tissue arrow into tissue at an offset location from the first implantation member.

22. The method according to claim 11, where the fibrous implant or transplant is a synthetic, polymeric mesh or scaffold, collagenous mesh or scaffold, periosteum transplant or a transplant including connective tissue.
